(19) United States
(12) Patent Application Publication
(10) Pub. No.: US 2025/0032579 A1
(43) Pub. Date: Jan. 30, 2025

(54) FORMULATION, AND PREPARATION METHOD THEREFOR AND USE THEREOF

(71) Applicants: JECHO BIOPHARMACEUTICALS CO., LTD., Tianjin (CN); JECHO LABORATORIES, INC., Frederick District, MD (US); JECHO INSTITUTE, CO., LTD., Shanghai (CN)

(72) Inventors: Jianwei ZHU, Tianjin (CN); Yueqing XIE, Tianjin (CN); Hua JIANG, Tianjin (CN); Zhenyu WANG, Tianjin (CN); Jin MA, Tianjin (CN); Lei HAN, Tianjin (CN)

(21) Appl. No.: 18/715,704

(22) PCT Filed: Dec. 2, 2022
(86) PCT No.: PCT/CN2022/136058

§ 371 Date: Jun. 1, 2024

(30) Foreign Application Priority Data
Dec. 3, 2021 (CN) 202111469687.X

(51) Int. Cl. A61K 38/18; A61K 9/00; A61K 9/08; A61K 47/02; A61K 47/26

(52) U.S. Cl. CPC A61K 38/1816; A61K 9/0019; A61K 9/08; A61K 47/02; A61K 47/26

(57) ABSTRACT

A formulation, comprising erythropoietin, a buffer ingredient, a surfactant and an osmotic pressure regulator. The present invention also relates to a method for preparing the formulation and the use of the formulation. The formulation is safe and stable and has a long validity period, and the preparation method is simple and easy to implement.

### TECHNICAL FIELD

The present application relates to the field of biomedicine, and specifically to a formulation as well as a preparation method and use thereof.

### BACKGROUND

Currently, erythropoietin can stimulate erythropoiesis (increase the red blood cell levels) and is commonly used to treat anemia caused by chronic kidney disease, malignant tumors and chemotherapy, etc. Long-acting erythropoiesis-stimulating protein is a type of erythropoietin. Compared with other marketed products, it has the advantages of long serum half-life and low dosing frequency.

Erythropoietin belongs to the cytokine family. Cytokines have a high affinity with cell surface receptors, making them highly effective molecules. Therefore, cytokines usually feature low dose and narrow therapeutic index. The low concentration of active ingredients not only challenges the protein structure analysis and stability research, but also reduces the therapeutic effect due to protein loss caused by protein adsorption or aggregation. At the same time, the physical and chemical instability of the protein itself may also lead to the formation of immunogenic degradation products, which may cause serious adverse reactions, bring great risks to clinical applications, and bring challenges to long-term storage.

The biological mechanisms and clinical applications of erythropoietin have been well reviewed, but less research has been done on its formula and stability. Improper formulation formula can easily cause chemical reactions such as deamidation, oxidation, aggregation, and degradation, and physical reactions such as protein adsorption, thereby reducing biological activity and increasing immunogenicity.

Therefore, it is particularly important to provide a safe, stable, and long-effective formulation and a preparation method thereof.

### SUMMARY OF THE INVENTION

The present application provides a formulation as well as a preparation method and use thereof. The formulation according to the present application can maintain good stability without comprising a stabilizer (e.g., betaine). The formulation according to the present application is safe, stable and has a long shelf life. The preparation method of the formulation is simple to operate and has high production efficiency.

In one aspect, the present application provides a formulation comprising erythropoietin, a buffer ingredient, a surfactant and an osmotic pressure regulator.

In certain embodiments, the erythropoietin comprises long-acting erythropoiesis-stimulating protein.

In certain embodiments, the erythropoietin comprises the amino acid sequences set forth in SEQ ID NO: 1.

In certain embodiments, the pH of the formulation is about 5.5 to about 7.0.

In certain embodiments, the buffer ingredient comprises a phosphate buffer solution, a citrate buffer solution and/or an acetate buffer solution.

In certain embodiments, the surfactant comprises a nonionic surfactant.

In certain embodiments, the surfactant comprises polysorbate.

In certain embodiments, the surfactant comprises polysorbate-80.

In certain embodiments, the osmotic pressure regulator comprises sodium chloride and/or mannitol.

In certain embodiments, the formulation comprises a solvent.

In certain embodiments, the solvent comprises pure water and/or water for injection.

In certain embodiments, the content of the long-acting erythropoiesis-stimulating protein is about 25 μg/mL to about 500 μg/mL.

In certain embodiments, the content of the long-acting erythropoiesis-stimulating protein is about 50 μg/mL to about 500 μg/mL.

In certain embodiments, the content of the buffer ingredient is about 0.001 mM to about 50 mM.

In certain embodiments, the buffer ingredient comprises a phosphate buffer solution, and the content of the phosphate buffer solution is about 0.001 mM to about 30 mM.

In certain embodiments, the buffer ingredient comprises a citrate buffer solution, and the content of the citrate buffer solution is about 0.001 mM to about 25 mM.

In certain embodiments, the buffer ingredient comprises an acetate buffer solution, and the content of the acetate buffer solution is about 0.001 mM to about 10 mM.

In certain embodiments, the surfactant comprises polysorbate-80, and the content of the polysorbate-80 is about 0.01 mg/mL to 0.1 mg/mL.

In certain embodiments, the osmotic pressure regulator comprises sodium chloride, and the content of the sodium chloride is about 100 mM to about 150 mM.

In certain embodiments, the osmotic pressure regulator comprises sodium chloride, and the content of the sodium chloride is about 5 mg/mL to about 10 mg/mL.

In certain embodiments, the content of the solvent is about 0.1 mL to about 10 mL.

In certain embodiments, the formulation comprises: 0.4 mM to 2.7 mM monobasic sodium phosphate monohydrate, 0.7 mM to 7 mM dibasic sodium phosphate, 0.01 mg/mL to 0.1 mg/mL polysorbate-80, 100 mM to 150 mM sodium chloride and 1 mL water for injection or purified water.

In certain embodiments, the formulation is formulated as an injection.

In certain embodiments, the storage temperature of the formulation is about 2 to about 8° C.

In another aspect, the present application provides a method for preparing the formulation according to the present application, the method comprising the following steps: 1) mixing the buffer ingredient, the osmotic pressure regulator and the surfactant to obtain a mixed solution; and 2) mixing the erythropoietin with the mixed solution.

In another aspect, the present application provides use of the formulation according to the present application in the preparation of erythropoietin drugs.

In another aspect, the present application provides use of the formulation according to the present application in the preparation of drugs for preventing and/or treating erythropoietin-related diseases.

In certain embodiments, the erythropoietin-related disease includes a red blood cell-related disease.

In certain embodiments, the erythropoietin-related disease includes anemia.

In certain embodiments, the erythropoietin-related disease includes anemia caused by kidney diseases, liver diseases, and/or tumors.

Those skilled in the art will readily appreciate other aspects and advantages of the present application from the detailed description below. Only exemplary embodiments of the present application are shown and described in the following detailed description. As those skilled in the art will realize, the contents of the present application enable those skilled in the art to make changes to the specific embodiments disclosed without departing from the spirit and scope of the invention covered by the present application. Accordingly, the drawings and descriptions in the specification of the present application are illustrative only and not restrictive.

### BRIEF DESCRIPTION OF DRAWINGS

The specific features of the invention to which the present application relates are set forth in the appended claims. The features and advantages of the invention to which the present application relates can be better understood by reference to the exemplary embodiments described in detail below and the drawings. A brief description of the drawings is as follows:

FIG. 1 shows the model fitting profile of the Plackett-Burman experimental design for the candidate formulations.

FIG. 2 shows the model fitting coefficient of the Plackett-Burman experimental design for the candidate formulations.

FIG. 3 shows the model fitting profile of the central composite experimental design for the candidate formulations.

FIG. 4 shows the contour analysis of the central composite experiment for the candidate formulations.

### DETAILED DESCRIPTION OF THE INVENTION

The embodiments of the invention of the present application will be described below with specific examples. Those skilled in the art can easily understand other advantages and effects of the invention of the present application from the disclosure of the specification.

### DEFINITION OF TERMS

In the present application, the term “erythropoietin” and its abbreviation “EPO” generally refer to any erythropoietin polypeptide, including but not limited to, erythropoietin polypeptides produced by recombination, erythropoietin polypeptides synthetically produced, natural EPO polypeptides, and erythropoietin polypeptides extracted from cells and tissues, the tissue including, but not limited to, kidney, liver, urine and blood. The biological activity produced after erythropoietin binds to the EPO receptor may include: administering erythropoietin to a subject by injection causes bone marrow cells to increase the production of reticulocytes and red blood cells compared to individuals in an uninjected group or a control group. For example, the erythropoietin may be erythropoietin having the amino acid sequence of SEQ ID No: 1. For example, the erythropoietin may be a variant protein having an amino acid sequence of SEQ ID No: 1, in which one or more amino acid residues are altered, deleted or inserted, and which has the same biological activity as the unmodified protein, such as reported in EP 1 064 951 or U.S. Pat. No. 6,583,272.

In the present application, the term “long-acting erythropoiesis-stimulating protein” generally refers to a protein which has a long stability and half-life as compared to the erythropoietin on the basis of retaining the original biological properties of the erythropoietin. The long-acting erythropoiesis-stimulating protein can have amino acid sequence and/or non-amino acid sequence modifications (such as modifications of glycosylation sites) based on the erythropoietin.

In the present application, the term “formulation” generally refers to a product comprising the erythropoietin according to the present application in a predetermined amount or proportion, and any product produced directly or indirectly by combination of a predetermined amount of the erythropoietin according to the present application. The formulation according to the present application may include pharmaceutical formulations, that is, include the erythropoietin according to the present application and excipients, as well as any product produced directly or indirectly by combining, compounding or aggregating any two or more ingredients. The formulation may be present in liquid form.

In the present application, the term “buffer ingredient” generally refers to agents having a function of providing a buffering effect (e.g., resistance to pH change). For example, the buffer ingredient can adjust changes in pH due to the addition and/or release of acidic or basic substances. For example, the buffer ingredient may include a weak acid and its conjugate base; or may include a weak base and its conjugate acid.

In the present application, the term “surfactant” generally refers to an agent that can protect a protein (e.g., the antigen-binding fragment according to the present application) from air/solution interface induced stress, solution/surface induced stress to reduce aggregation of the protein or minimize formation of particulate matter in the composition. The surfactant may comprise both hydrophobic groups and hydrophilic groups. The surfactant may include a mixture or combination of one or more surfactants. The surfactant may include a nonionic surfactant.

In the present application, the term “non-ionic surfactant” generally refers to a surfactant comprising a hydrophilic group that is not dissociated in an aqueous solution. For example, the nonionic surfactant may not be dissociated into an ionic state in an aqueous solution, but may exist in the solution in a molecular or micellar state. For example, the nonionic surfactant may include polyoxyethylene type nonionic surfactants (e.g., fatty alcohol polyoxyethylene ether, alkylphenol polyoxyethylene ether (OP), octylphenol polyoxyethylene ether-10, dodecylphenol polyoxyethylene ether or fatty acid polyoxyethylene ether), polyol type inactive surfactants (e.g., Span type surfactants, Tween type surfactants) and/or polyethers (e.g., full-block polyethers). The nonionic surfactant may be highly stable, less affected by acids, bases, and ions, and/or have strong resistance to hard water.

In the present application, the term “osmotic pressure regulator” generally refers to an agent used to regulate the osmotic pressure of a liquid formulation. For example, the osmotic pressure regulator can make the osmotic pressure of a liquid formulation containing the agent isotonic with body fluids (e.g., human plasma) to avoid damaging tissues. In the present application, the osmotic pressure regulator may include sodium chloride, glycerol, glucose and/or mannitol.

In the present application, the term “solvent” generally refers to a liquid that has the ability to dissolve other substances. The solvent may include organic solvents (e.g., aromatic hydrocarbons, aliphatic hydrocarbons, alicyclic hydrocarbons, halogenated hydrocarbons, alcohols, ethers, esters, or ketones) and inorganic solvents (e.g., water). For example, the water may include pure water, i.e., H₂O without impurities. For example, the water may include water for injection, i.e., water that complies with the provisions of the Water for Injection item of the Chinese Pharmacopoeia. The water for injection may include distilled water or water obtained by distilling deionized water.

In the present application, the term “red blood cell-related diseases” generally refers to diseases caused by changes in the number, morphology and/or nature of red blood cells. For example, the red blood cell-related diseases may include: diseases related to red blood cell synthesis disorders (such as iron deficiency (ID), iron deficiency anemia (IDA), sideroblastic anemia (SA), megaloblastic anemia (MA)), genetic-related hemolytic anemia (such as red cell enzymopathies, hemoglobin disease, hereditary spherocytosis (HS), hereditary elliptocytosis (HE), hereditary stomatocytosis, acanthocytosis) and/or hemolytic anemia (such as autoimmune hemolytic anemia (AIHA), maternal-fetal blood group incompatibility hemolytic disease (BGIHD), drug-induced hemolytic anemia), as well as anemia of chronic disease (ACD), pure red cell aplasia (PRCA) or myelophthisic anemia (MTA).

In the present application, the term “anemia” generally refers to a common clinical symptom in which the red blood cell volume in the peripheral blood of a human body is reduced below the lower limit of the normal range. For example, the anemia may include anemia due to decreased erythropoiesis, anemia caused by abnormal hematopoietic microenvironment (e.g., anemia caused by damaged bone marrow stroma and stromal cells, anemia caused by abnormal levels of hematopoietic regulatory factors, anemia caused by hyperfunction of lymphocytes or hyperapoptosis of hematopoietic cells), anemia due to excessive destruction of red blood cells, and hemorrhagic anemia (e.g., coagulation diseases (e.g., hemophilia, liver disease, and chronic kidney disease) or non-coagulation diseases (e.g., tumors, tuberculosis, or peptic ulcer)).

In the present application, the term “about” generally refers to the usual margin of error of the corresponding value. Reference herein to “about” a value or parameter includes (and describes) options for that value or parameter per se. If in doubt, or if the error range for a particular value or parameter is not generally understood as recognized in the art, “about” means±5% of the value or parameter.

### DETAILED DESCRIPTION OF THE INVENTION

In one aspect, the present application provides a formulation comprising erythropoietin, a buffer ingredient, a surfactant and an osmotic pressure regulator.

In the present application, the erythropoietin may include long-acting erythropoiesis-stimulating protein.

For example, the long-acting erythropoiesis-stimulating protein may comprise an N-glycosylation site. For example, the N-glycosylation site may include N24, N30, N38, N83 and/or N88.

For example, the long-acting erythropoiesis-stimulating protein may contain a glycan structure bound to an N-glycosylation site, and the glycan structure comprises FA4G4L2S4, wherein F represents fucose, A represents N-acetylglucosamine, G represents galactose, L represents lactose, and S represents sialic acid. For example, the proportion of the FA4G4L2S4 structure is greater than 15%. For example, the glycan structure may comprise FA4G4L1S4, wherein F represents fucose, A represents N-acetylglucosamine, G represents galactose, L represents lactose, and S represents sialic acid. For example, the proportion of FA4G4L1S4 may be greater than 20%. For example, the glycan structure comprises FA4G4S4, wherein F represents fucose, A represents N-acetylglucosamine, G represents galactose, and S represents sialic acid. For example, the proportion of the FA4G4S4 is greater than 10%. For example, the glycan structure comprises Neu5Gc, and the molar proportion of Neu5Gc is 0.5% or less.

In the present application, the erythropoietin may comprise the amino acid sequences set forth in SEQ ID NO: 1.

In the present application, the pH of the formulation may be about 5.5 to about 7.0. For example, the pH of the formulation can be about 6.0 to about 7.0, about 6.5 to about 7.0, about 5.5 to about 6.5, or about 5.5 to about 6.0.

In the present application, the buffer ingredient may include a phosphate buffer solution, a citrate buffer solution and/or an acetate buffer solution. For example, the phosphate buffer solution may include monobasic sodium phosphate and dibasic sodium phosphate. The citrate buffer solution may include citric acid and sodium hydrogen phosphate. The acetate buffer solution may include ammonium acetate and hydrochloric acid.

In the present application, the surfactant may include a nonionic surfactant.

In the present application, the surfactant may include polysorbate. For example, the surfactant may include polysorbate-80.

In the present application, the osmotic pressure regulator may include sodium chloride and/or mannitol.

In the present application, the formulation may include a solvent. For example, the solvent may include purified water and/or water for injection.

In the present application, the content of the long-acting erythropoiesis-stimulating protein may be about 25 μg/mL to about 500 μg/mL. For example, it may be about 25 μg/mL to about 450 μg/mL, about 25 μg/mL to about 400 μg/mL, about 25 μg/mL to about 350 μg/mL, about 25 μg/mL to about 300 μg/mL, about 25 μg/mL to about 250 μg/mL, about 25 μg/mL to about 200 μg/mL, about 25 μg/mL to about 150 μg/mL, about 25 μg/mL to about 100 g/mL, about 25 μg/mL to about 50 μg/mL, about 30 μg/mL to about 500 μg/mL, about 50 g/mL to about 500 μg/mL, about 100 μg/mL to about 500 μg/mL, about 150 μg/mL to about 500 μg/mL, about 200 μg/mL to about 500 μg/mL, about 250 μg/mL to about 500 μg/mL, about 300 μg/mL to about 500 μg/mL, about 350 μg/mL to about 500 μg/mL, or about 400 μg/mL to about 500 μg/mL. For example, the content of the long-acting erythropoiesis-stimulating protein may be about 50 μg/mL to about 500 μg/mL.

In the present application, the content of the buffer ingredient may be about 0.001 mM to about 50 mM. For example, it may be about 0.001 mM to about 40 mM, about 0.001 mM to about 35 mM, about 0.001 mM to about 30 mM, about 0.001 mM to about 25 mM, about 0.001 mM to about 10 mM, about 0.001 mM to about 5 mM, about 0.001 mM to about 1 mM, about 0.001 mM to about 0.1 mM, about 0.001 mM to about 0.01 mM, about 0.01 mM to about 50 mM, about 0.05 mM to about 50 mM, about 0.1 mM to about 50 mM, or about 1 mM to about 50 mM.

In the present application, the buffer ingredient may include a phosphate buffer solution, and the content of the phosphate buffer solution may be about 0.001 mM to about 30 mM. For example, it may be about 0.001 mM to about 29 mM, about 0.001 mM to about 28 mM, about 0.001 mM to about 27 mM, about 0.001 mM to about 26 mM, about 0.001 mM to about 25 mM, about 0.001 mM to about 20 mM, about 0.001 mM to about 15 mM, about 0.001 mM to about 10 mM, about 0.001 mM to about 7 mM, about 0.001 mM to about 1 mM, about 0.1 mM to about 27 mM, about 0.1 mM to about 20 mM, about 0.4 mM to about 30 mM, about 0.4 mM to about 27 mM, about 0.4 mM to about 7 mM, about 0.7 mM to about 27 mM, or about 0.7 mM to about 7 mM.

In the present application, the buffer ingredient may include a citrate buffer solution, and the content of the citrate buffer solution may be about 0.001 mM to about 25 mM. For example, it may be about 0.001 mM to about 20 mM, about 0.001 mM to about 15 mM, about 0.001 mM to about 10 mM, about 0.001 mM to about 5 mM, about 0.001 mM to about 1 mM, about 0.01 mM to about 25 mM, about 0.01 mM to about 20 mM, about 0.01 mM to about 15 mM, about 0.01 mM to about 10 mM, about 0.01 mM to about 5 mM, about 0.01 mM to about 1 mM or about 0.001 mM to about 1 mM.

In the present application, the buffer ingredient may include an acetate buffer solution, and the content of the acetate buffer solution may be about 0.001 mM to about 10 mM. For example, it may be about 0.001 mM to about 8 mM, about 0.001 mM to about 6 mM, about 0.001 mM to about 4 mM, about 0.001 mM to about 2 mM, about 0.001 mM to about 1 mM, about 0.01 mM to about 10 mM, about 0.01 mM to about 8 mM, about 0.01 mM to about 6 mM, about 0.01 mM to about 4 mM, about 0.01 mM to about 2 mM, about 0.01 mM to about 1 mM or about 0.001 mM to about 1 mM.

In the present application, the surfactant may include polysorbate-80, and the content of the polysorbate-80 may be about 0.001% to about 0.01%. For example, it may be about 0.001% to about 0.009%, about 0.001% to about 0.008%, about 0.001% to about 0.007%, about 0.001% to about 0.006%, about 0.001% to about 0.005%, about 0.002% to about 0.01%, about 0.002% to about 0.009%, about 0.002% to about 0.008%, about 0.002% to about 0.007%, about 0.002% to about 0.006%, or about 0.002% to about 0.05%.

In the present application, the surfactant may include polysorbate-80, and the content of the polysorbate-80 may be about 0.01 mg/mL to about 0.1 mg/mL. For example, it may be about 0.01 mg/mL to 0.09 mg/mL, about 0.01 mg/mL to 0.08 mg/mL, about 0.01 mg/mL to 0.07 mg/mL, about 0.01 mg/mL to 0.06 mg/mL, about 0.01 mg/mL to 0.05 mg/mL, about 0.01 mg/mL to 0.04 mg/mL, about 0.01 mg/mL to 0.1 mg/mL.

In the present application, the osmotic pressure regulator may include sodium chloride, and the content of the sodium chloride may be about 100 mM to about 150 mM. For example, it may be about 110 mM to about 150 mM, about 115 mM to about 150 mM, about 120 mM to about 150 mM, about 125 mM to about 150 mM, about 130 mM to about 150 mM, about 135 mM to about 150 mM, or about 100 mM to about 140 mM.

In the present application, the osmotic pressure regulator may include sodium chloride, and the content of the sodium chloride may be about 5 mg/mL to about 10 mg/mL. For example, it may be about 5.5 mg/mL to about 10 mg/mL, about 6 mg/mL to about 10 mg/mL, about 6.5 mg/mL to about 10 mg/mL, about 7 mg/mL to about 10 mg/mL, about 7.5 mg/mL to about 10 mg/mL, about 5 mg/mL to about 9 mg/mL, about 6 mg/mL to about 9 mg/mL, or about 5 mg/mL to about 8 mg/mL.

In the present application, the content of the solvent may be about 0.1 mL to about 10 mL. For example, it may be about 0.1 mL to about 10 mL, about 0.1 mL to about 9 mL, about 0.1 mL to about 8 mL, about 0.1 mL to about 7 mL, about 0.1 mL to about 6 mL, about 0.1 mL to about 5 mL, about 0.5 mL to about 10 mL, about 1 mL to about 10 mL, about 2 mL to about 10 mL, or about 2 mL to about 10 mL.

In the present application, the formulation may comprise erythropoietin, a buffer ingredient, a surfactant, an osmotic pressure regulator and a solvent.

In the present application, the formulation may consist of erythropoietin, a buffer ingredient, a surfactant, an osmotic pressure regulator and a solvent.

In the present application, the formulation may not comprise a stabilizer.

In the present application, the formulation may comprise: 0.4 mM to 2.7 mM monobasic sodium phosphate monohydrate, 0.7 mM to 7 mM dibasic sodium phosphate, 0.01 mg/mL to 0.1 mg/mL polysorbate-80, 100 mM to 150 mM sodium chloride and 1 mL water for injection or purified water.

In the present application, the formulation may comprise: about 25 μg/mL to about 500 μg/mL erythropoiesis-stimulating protein, 0.4 mM to 2.7 mM monobasic sodium phosphate monohydrate, 0.7 mM to 7 mM dibasic sodium phosphate, 0.01 mg/mL to 0.1 mg/mL polysorbate-80, 100 mM to 150 mM sodium chloride and 1 mL water for injection or purified water.

In the present application, the formulation may consist of about 25 μg/mL to about 500 μg/mL erythropoiesis-stimulating protein, 0.4 mM to 2.7 mM monobasic sodium phosphate monohydrate, 0.7 mM to 7 mM dibasic sodium phosphate, 0.01 mg/mL to 0.1 mg/mL polysorbate-80, 100 mM to 150 mM sodium chloride and water for injection or purified water.

In the present application, the formulation may comprise: 0.6 mg/mL to 3.7 mg/mL monobasic sodium phosphate monohydrate, 0.1 mg/mL to 1 mg/mL dibasic sodium phosphate, 0.01 mg/mL to 0.1 mg/mL polysorbate-80, 5.85 mg/mL to 8.76 mg/mL sodium chloride and 1 mL water for injection or purified water.

In the present application, the formulation may consist of erythropoiesis-stimulating protein (50.0 μg/mL), 0.6 mg/mL to 3.7 mg/mL monobasic sodium phosphate monohydrate, 0.1 mg/mL to 1 mg/mL dibasic sodium phosphate, 0.01 mg/mL to 0.1 mg/mL polysorbate-80, 5.85 mg/mL to 8.76 mg/mL sodium chloride and 1 mL water for injection or purified water.

In the present application, the formulation may comprise long-acting erythropoiesis-stimulating protein (about 50 μg/mL to about 500 μg/mL) and a phosphate buffer (about 15 mM to about 50 mM), sodium chloride (about 100 mM to about 140 mM), polysorbate 80 (about 0.005% to about 0.01%), and water for injection or purified water.

In the present application, the formulation may comprise long-acting erythropoiesis-stimulating protein (50.0 μg/mL) and a phosphate buffer (15 mM), sodium chloride (140 mM), polysorbate 80 (0.005%), and water for injection or purified water.

In the present application, the formulation may consist of long-acting erythropoiesis-stimulating protein (50.0 μg/mL) and a phosphate buffer (15 mM), sodium chloride (140 mM), polysorbate 80 (0.005%), and water for injection or purified water.

In that present application, the formulation may be formulated as an injection. For example, the injection can be administered by intravenous injection.

In the present application, the storage temperature of the formulation may be about 2 to about 8° C. For example, the formulation can be stored frozen at about 4° C.

In another aspect, the present application provides a method for preparing the formulation according to the present application, and the method may comprise the following steps:
1) mixing the buffer ingredient, the osmotic pressure regulator and the surfactant to obtain a mixed solution; and 2) mixing the erythropoietin with the mixed solution.

In the preparation method of the present application, there is no mandatory requirement for the order of adding the buffer ingredient, the osmotic pressure regulator, the surfactant and erythropoietin, as long as the above ingredients are fully mixed. The above ingredients can be fully dissolved in the solvent of the present application after mixing to form a uniform composition.

In another aspect, the present application provides use of the formulation according to the present application in the preparation of erythropoietin drugs.

In another aspect, the present application provides use of the formulation according to the present application in the preparation of drugs for preventing and/or treating erythropoietin-related diseases.

In certain embodiments, the erythropoietin-related disease may include a red blood cell-related disease.

In certain embodiments, the erythropoietin-related disease may include anemia.

In certain embodiments, the erythropoietin-related disease may include anemia caused by kidney diseases, liver diseases, and/or tumors.

Without wishing to be bound by any theory, the following Examples are only for illustrating the fusion protein, preparation method and use of the present application, and are not used to limit the scope of the invention of the present application.

### Examples

### Example 1 Screening of Significant Factors for the Stability of Long-Acting Erythropoiesis-Stimulating Protein

The importance of the ingredients of the formula on the stability of long-acting erythropoiesis-stimulating protein was evaluated using the Plackett-Burman experimental design. Significant influencing factors were screened for the buffer system (phosphate buffer, citrate buffer, and acetate buffer), pH (6.0-7.0), polysorbate 80 (0.001% to 0.01%), and sodium chloride (100-150 mM). An experimental design with the number of experiments N=12 was used, with 3 central points added, constituting a total of 15 groups of experiments. The factors and levels of the Plackett-Burman experimental design are shown in Table 1.

TABLE 1
Variables and levels of Plackett-Burman experimental design

| | | |
|---|---|---|
| Variable | −1 | +1 |

| | | | |
|---|---|---|---|
| Buffer system | Phosphate buffer | 0 | 25 mM |
| | Citrate buffer | 0 | 25 mM |
| | Acetate buffer | 0 | 10 mM |

| | | |
|---|---|---|
| pH | 6 | 7 |
| Sodium chloride | 100 mM | 150 mM |
| Polysorbate 80 | 0.001% | 0.01% |

The Plackett-Burman experimental design and response values performed using MODDE software (Sartorius data analysis software version 12) are shown in Table 2.

The biophysical detection methods used include: differential scanning calorimetry (DSC) for analyzing the conformational stability and thermodynamic stability of the samples; size exclusion chromatography (SEC-HPLC) for detecting changes in the content of polymers, monomers and fragments; and cell assays for detecting in vitro biological activity of the samples. Melting temperature (Tm), SEC purity and in vitro biological activity measurements were used as response factors.

The long-acting erythropoiesis-stimulating protein stock solution (wherein the long-acting erythropoiesis-stimulating protein comprises the amino acid sequence set forth in SEQ ID NO.1) was concentrated and displaced into the 15 experimental combinations listed in Table 2 at 2-8° C., and the concentration of the long-acting erythropoiesis-stimulating protein was adjusted to 200 μg/mL (+10%). Freshly prepared samples of the 15 experimental combinations were taken to detect the melting temperature value; the 15 groups of samples were then placed in vials and treated under accelerated conditions (50° C.) for 8 days, and then the changes in the contents of polymers, monomers and fragments were detected by size exclusion chromatography; and the changes in the in vitro biological activity of the samples were analyzed by cell assays.

TABLE 2
Plackett-Burman experimental design and response values

| | | | | | | | | | | | | Changes in |
|---|---|---|---|---|---|---|---|---|---|---|---|---|
| | | | | Phosphate | Citrate | Acetate | | | Polysorbate | | SEC- | biological |
| | | | Incl/ | buffer | buffer | buffer | | NaCl | 80 | Tm | HPLC | activity |
| No. | Name | Order | Excl | (mM) | (mM) | (mM) | pH | (mM) | (%) | (° C.) | (%) | (%) |
| 1 | N1 | 14 | Incl | 25 | 0 | 10 | 6 | 100 | 0.001 | 47.88 | 94.52 | 34.29 |
| 2 | N2 | 4 | Incl | 25 | 25 | 0 | 7 | 100 | 0.001 | 52.87 | 78.85 | 33.64 |
| 3 | N3 | 7 | Incl | 0 | 25 | 10 | 6 | 150 | 0.001 | 51.02 | 93.98 | 33.64 |
| 4 | N4 | 13 | Incl | 25 | 0 | 10 | 7 | 100 | 0.01 | 49.33 | 66.81 | 18.72 |
| 5 | N5 | 5 | Incl | 25 | 25 | 0 | 7 | 150 | 0.001 | 53.04 | 70.89 | 26.95 |
| 6 | N6 | 6 | Incl | 25 | 25 | 10 | 6 | 150 | 0.01 | 50.38 | 92.70 | 42.47 |
| 7 | N7 | 3 | Incl | 0 | 25 | 10 | 7 | 100 | 0.01 | 50.09 | 58.91 | 14.33 |
| 8 | N8 | 10 | Incl | 0 | 0 | 10 | 7 | 150 | 0.001 | 49.15 | 47.42 | 2.83 |
| 9 | N9 | 9 | Incl | 0 | 0 | 0 | 7 | 150 | 0.01 | 47.77 | 45.07 | 1.96 |
| 10 | N10 | 2 | Incl | 25 | 0 | 0 | 6 | 150 | 0.01 | 47.44 | 96.39 | 39.88 |
| 11 | N11 | 11 | Incl | 0 | 25 | 0 | 6 | 100 | 0.01 | 49.23 | 94.23 | 34.67 |
| 12 | N12 | 12 | Incl | 0 | 0 | 0 | 6 | 100 | 0.001 | 45.72 | 57.08 | 6.37 |
| 13 | N13 | 8 | Incl | 12.5 | 12.5 | 5 | 6.5 | 125 | 0.0055 | | 86.20 | 27.94 |
| 14 | N14 | 1 | Incl | 12.5 | 12.5 | 5 | 6.5 | 125 | 0.0055 | 49.87 | 86.88 | 24.57 |
| 15 | N15 | 15 | Incl | 12.5 | 12.5 | 5 | 6.5 | 125 | 0.0055 | 50.02 | 85.71 | 29.98 |

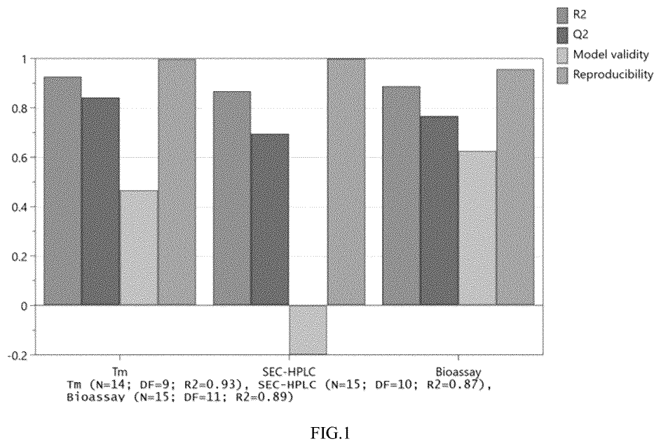

From the model fitting profile (FIG. 1), it can be seen that when fitting is performed with the melting temperature as the response, the model established with the melting temperature as the response works well.

When fitting is performed with SEC purity as the response, it can be considered that the model established with SEC purity as the response works well.

When fitting is performed with in vitro biological activity as the response, the model established with in vitro biological activity as the response works well.

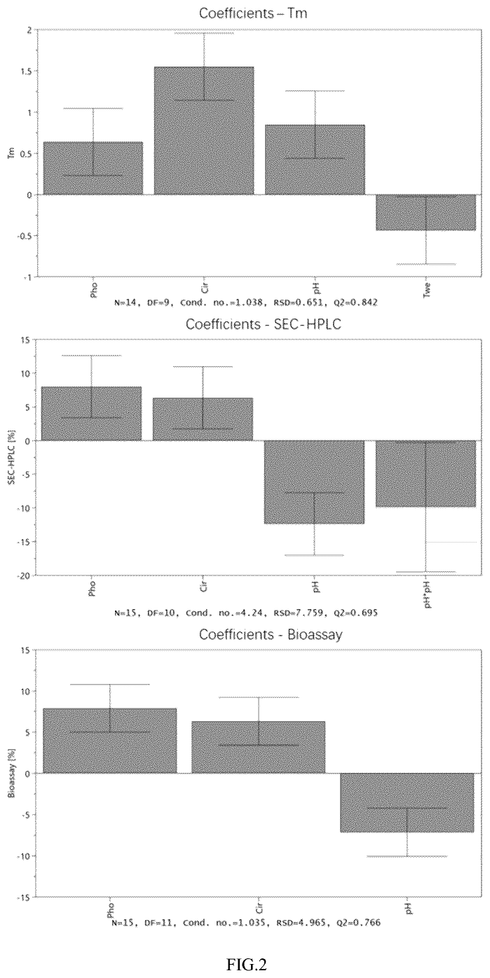

According to the model fitting coefficient (FIG. 2), phosphate buffer and pH are significant influencing factors.

### Example 2 Optimization of the Formula Composition of Long-Acting Erythropoiesis-Stimulating Protein

To obtain the formula composition of long-acting erythropoiesis-stimulating protein, the formula of long-acting erythropoiesis-stimulating protein was optimized using the central composite experimental design (CCF) based on the Plackett-Burman experiment. The Plackett-Burman experiment identified phosphate buffer and pH as significant influencing factors. Each significant influencing factor was studied at three levels: low, medium, and high. The factors and levels of the central composite experimental design are shown in Table 3.

TABLE 3
Variables and levels of central composite design

| Variable | −1 | 0 | +1 |
|---|---|---|---|
| Phosphate concentration | 15 | 20 | 25 |
| pH | 5.5 | 6 | 6.5 |

The central composite experimental design was performed using MODDE software (Sartorius data analysis software version 12) with the number of experiments N=8, with 3 central points added. Each experiment had 2 replicates, and there were 22 experiments in total. The experimental design and response values are shown in Table 4.

Melting temperature values and SEC-HPLC purity were used as response values. The long-acting erythropoiesis-stimulating protein stock solution (wherein the long-acting erythropoiesis-stimulating protein comprises the amino acid sequence set forth in SEQ ID NO.1) was concentrated and displaced into the 22 combinations listed in Table 4 at 2-8° C., and the concentration of the long-acting erythropoiesis-stimulating protein was adjusted to 200 μg/mL (+10%). Freshly prepared samples were taken to detect the melting temperature values of the 22 combinations; then the 22 groups of samples were treated under accelerated conditions (50° C.) for 8 days, and then the changes in the contents of polymers, monomers and fragments were detected by size exclusion chromatography.

TABLE 4
Central composite experimental design and response values

| | | | | Phosphate | | SEC- | |
|---|---|---|---|---|---|---|---|
| | | | Incl/ | buffer | | HPLC | Tm |
| No. | Name | Order | Excl | (mM) | pH | (%) | (° C.) |
| 1 | N1 | 21 | Incl | 15 | 5.5 | 96.72 | 38.50 |
| 2 | N2 | 8 | Incl | 25 | 5.5 | 94.78 | 38.90 |
| 3 | N3 | 17 | Incl | 15 | 6.5 | 93.29 | 44.48 |
| 4 | N4 | 10 | Incl | 25 | 6.5 | 92.82 | 45.59 |
| 5 | N5 | 18 | Incl | 15 | 6 | 96.14 | 42.57 |
| 6 | N6 | 7 | Incl | 25 | 6 | 95.52 | 42.42 |
| 7 | N7 | 16 | Incl | 20 | 5.5 | 95.75 | 40.18 |
| 8 | N8 | 4 | Incl | 20 | 6.5 | 93.79 | 44.28 |
| 9 | N9 | 3 | Incl | 20 | 6 | 95.90 | 42.84 |
| 10 | N10 | 6 | Incl | 20 | 6 | 95.55 | 43.11 |
| 11 | N11 | 20 | Incl | 20 | 6 | 95.55 | 42.64 |
| 12 | N12 | 2 | Incl | 15 | 5.5 | 96.44 | 39.70 |
| 13 | N13 | 9 | Incl | 25 | 5.5 | 94.29 | 39.38 |
| 14 | N14 | 19 | Incl | 15 | 6.5 | 93.62 | 43.61 |
| 15 | N15 | 15 | Incl | 25 | 6.5 | 92.22 | 44.00 |
| 16 | N16 | 5 | Incl | 15 | 6 | 95.98 | 42.74 |
| 17 | N17 | 1 | Incl | 25 | 6 | 96.50 | 42.72 |
| 18 | N18 | 14 | Incl | 20 | 5.5 | 94.55 | 38.77 |
| 19 | N19 | 13 | Incl | 20 | 6.5 | 92.77 | 43.65 |
| 20 | N20 | 22 | Incl | 20 | 6 | 96.36 | 42.40 |
| 21 | N21 | 11 | Incl | 20 | 6 | 96.44 | 42.74 |
| 22 | N22 | 12 | Incl | 20 | 6 | 96.48 | 42.77 |

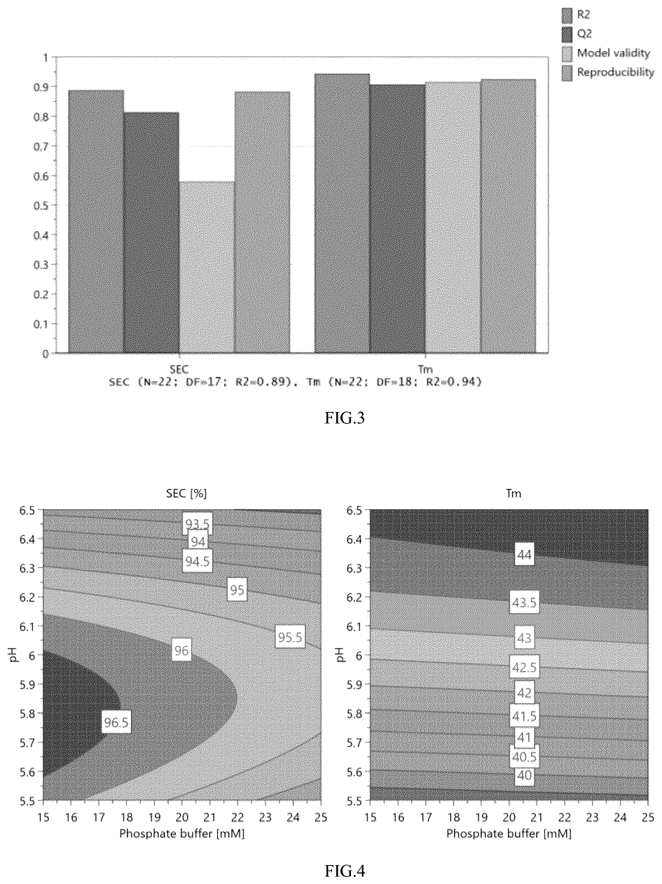

From the model fitting profile (FIG. 3), it can be seen that the model established with SEC purity as the response works well.

When fitting is performed with the melting temperature as the response, the model established with the melting temperature as the response works well.

High SEC-HPLC purity and melting temperature values indicate that long-acting erythropoiesis-stimulating protein is more stable in the formula composition. The model obtained by using the central composite experimental contour analysis (FIG. 4) predicts the formula with the highest values of both SEC-HPLC purity and melting temperature. The conclusion is as follows: when the phosphate buffer has a concentration of 15 mM and pH 6.1, the predicted SEC-HPLC purity is 96.18% (95.70%-96.66%), and the predicted melting temperature is 43.0 (42.5-43.5).

The formula composition with the concentration of long-acting erythropoiesis-stimulating protein being 200 μg/mL, the concentration of phosphate buffer being 15 mM, the concentration of sodium chloride being 125 mM, the concentration of polysorbate 80 being 0.005% at pH 6.1, was verified. After treatment at 50° C. for 8 days, the detection result of size exclusion chromatography was 95.84%, which was consistent with the model prediction result (95.70%-96.66%).

### Example 3 Determination of the Osmotic Pressure of an Injection

According to the provisions of Part III of the 2020 edition of the “Chinese Pharmacopeia”, the osmotic pressure of injections should be consistent with the blood osmotic pressure, ranging from 285 to 310 mOsmol/kg. The injection method adopted in the present application is subcutaneous injection or intravenous injection, so the osmotic pressure needs to meet the requirements of the pharmacopeia.

The osmotic pressure of the salt solution in the formula composition involved in the response surface experiment was determined using an ice point osmotic pressure detector (Loser, OM150).

The phosphate concentration is 15 mM, the sodium chloride concentration is 125 mM, and the buffer osmotic pressure at pH 6.1 is 262 mOsmol/kg, which is lower than the blood osmotic pressure range of 285-310 mOsmol/kg. The sodium chloride concentration was adjusted to 140 mM, and the other ingredients remained unchanged: the phosphate concentration was 15 mM, the polysorbate 80 concentration was 0.005%, and the osmotic pressure of the buffer solution with pH 6.1 was approximately 298 mOsm/kg, which met the requirements for the osmotic pressure of injections in Part III of the 2020 edition of the “Chinese Pharmacopoeia”.

### Example 4 Effect of Protein Concentration on the Stability of Long-Acting Erythropoiesis-Stimulating Protein

The investigation range of long-acting erythropoiesis-stimulating protein concentration was set to be 25 μg/mL, 50 μg/mL, 100 μg/mL, 200 μg/mL, 300 μg/mL and 500 μg/mL. The components of the buffer solution included: phosphate buffer at a concentration of 15 mM, and 140 mM sodium chloride. The long-acting erythropoiesis-stimulating protein stock solution (wherein the long-acting erythropoiesis-stimulating protein comprises the amino acid sequence set forth in SEQ ID NO.1) was concentrated and displaced to the set protein concentration at 2-8° C., respectively, and then polysorbate 80 at a concentration of 1% was added to reach a final concentration of 0.005%. After 8 days in a water bath at 50° C., the purity was detected by size exclusion chromatography.

The results of size exclusion chromatography of samples with different concentrations of long-acting erythropoiesis-stimulating protein: 25 μg/mL, 50 μg/mL, 100 μg/mL, 200 μg/mL, 300 μg/mL and 500 μg/mL after being incubated in a 50° C. water bath for 8 days are shown in Table 5.

TABLE 5
Size exclusion chromatography results for different
protein concentrations at 50° C. for 8 days

| Protein concentration (μg/mL) | SEC-HPLC purity (%) |
|---|---|
| 25 | 98.99 |
| 50 | 97.85 |
| 100 | 97.74 |
| 200 | 97.26 |
| 300 | 96.08 |
| 500 | 95.69 |

From the experimental results, it can be seen that the lower the protein concentration is, the higher the SEC-HPLC purity is. When the protein concentration is 25 μg/mL, the SEC-HPLC purity is the highest, which is 98.99%. There is no significant difference in the SEC-HPLC purity of samples with protein concentrations of 50 μg/mL, 100 μg/mL, 200 μg/mL, 300 μg/mL and 500 μg/mL.

After different concentrations of long-acting erythropoiesis-stimulating protein were placed in a 50° C. water bath for 8 days in the buffer of optimized formulation formula (15 mM phosphate, 140 mM sodium chloride, 0.005% polysorbate 80, pH 6.1), there was no significant difference in the SEC-HPLC purity of 25 μg/mL, 50 μg/mL, 100 μg/mL and 200 μg/mL samples, indicating that within the range of 25 μg/mL-200 μg/mL, protein concentration has no significant effect on SEC-HPLC purity.

In summary, the formula of long-acting erythropoiesis-stimulating protein injection formulation consists of long-acting erythropoiesis-stimulating protein (50.0 μg/mL) and phosphate buffer (15 mM), sodium chloride (140 mM), polysorbate 80 (0.005%) and water for injection, at pH 6.1, and it is called JL14001 finished product.

### Example 5 Preparation of Long-Acting Erythropoiesis-Stimulating Protein Formula

Formulation formula (per liter): 1.82 g of monobasic sodium phosphate monohydrate, 0.50 g of dibasic sodium phosphate, 8.18 g of sodium chloride, and 0.05 g of polysorbate 80.

### Preparation:

Solution No. 1: All components except polysorbate 80 were weighed according to the formula, 500 mL of injection water cooled to room temperature was added, the mixture was stirred to dissolve, mixed uniformly, made up to 1000 mL with water for injection, and stirred and mixed uniformly.

Solution No. 2:1.00 g of polysorbate 80 was weighed and, 50 mL of solution No. 1 was added, and the mixture was stirred gently to fully dissolve it while avoiding foaming. After 0093-PA-025US 21 mixing uniformly, the system was diluted to 100 mL with solution No. 1, gently stirred and mixed uniformly, and filtered aseptically to obtain a 1% polysorbate 80 solution.

Formulation solution: 398 mL of solution No. 1 was measured, 2 mL of solution No. 2 was added, and the mixture was filtered aseptically to obtain 400 mL of a formulation solution.

The long-acting erythropoiesis-stimulating protein stock solution (wherein the long-acting erythropoiesis-stimulating protein comprises the amino acid sequence set forth in SEQ ID NO.1) was diluted with prepared formulation solution so that the final concentration of the long-acting erythropoiesis-stimulating protein reached 300 μg/mL.

### Example 6 Test of Stability of Long-Acting Erythropoiesis-Stimulating Protein Formula

### 6.1 Long-Term Stability Test

### Long-Term Stability Data of Upright Samples

The results for the statistical data of the long-term (upright) stability test of JL14001 finished product at 2-8° C. (batch number: 04202102001) are shown in Table 6.

TABLE 6

| Test item | Standard | | Point 0 | 3 months |
|---|---|---|---|---|
| pH value | 5.9-6.3 | | 6.0 | 6.2 |
| Osmotic molar | 270-350 | mOsmol/kg | 294 | N/A |
| concentration | | | | |
| Protein content | 45-55 | μg/ml | 51 | 50 |

| | | | |
|---|---|---|---|
| HPLC purity | ≥98.0% | 100.0% | 100.0% |
| In vitro biological | 50%-150% of reference | 138% | 132% |
| activity | | | |

The results for the statistical data of the long-term (upright) stability test of JL14001 finished product at 2-8° C. (batch number: 04202102002) are shown in Table 7.

TABLE 7

| Test item | Standard | | Point 0 | 3 months |
|---|---|---|---|---|
| pH value | 5.9-6.3 | | 6.0 | 6.2 |
| Osmotic molar | 270-350 | mOsmol/kg | 294 | N/A |
| concentration | | | | |
| Protein content | 45-55 | μg/ml | 51 | 50 |

| | | | |
|---|---|---|---|
| HPLC purity | ≥98.0% | 100.0% | 100.0% |
| In vitro biological | 50%-150% of reference | 138% | 118% |
| activity | | | |

The results for the statistical data of the long-term (upright) stability test of JL14001 finished product at 2-8° C. (batch number: 04202102003) are shown in Table 8.

TABLE 8

| Test item | Standard | | Point 0 | 3 months |
|---|---|---|---|---|
| pH value | 5.9-6.3 | | 6.0 | 6.1 |
| Osmotic molar | 270-350 | mOsmol/kg | 294 | N/A |
| concentration | | | | |
| Protein content | 45-55 | μg/ml | 51 | 50 |

| | | | |
|---|---|---|---|
| HPLC purity | ≥98.0% | 100.0% | 100.0% |
| In vitro biological | 50%-150% of reference | 138% | 103% |
| activity | | | |

The results for statistical data of the long-term (upright) stability test of JL14001 finished product at 2-8° C. (batch number: 04202102003) are shown in Table 9.

TABLE 9

| Test item | Standard | | Point 0 | 3 months |
|---|---|---|---|---|
| pH value | 5.9-6.3 | | 6.0 | 6.2 |
| Osmotic molar | 270-350 | mOsmol/kg | 294 | N/A |
| concentration | | | | |
| Protein content | 45-55 | μg/ml | 51 | 51.0 |

| | | | |
|---|---|---|---|
| HPLC purity | ≥98.0% | 100.0% | 100.0% |
| In vitro biological | 50%-150% of reference | 138% | 131% |
| activity | | | |

### 6.1 Accelerated Stability Test

### Accelerated Stability Data of Upright Samples

The results for the statistical data of the accelerated stability test of JL14001 finished product at 25° C. (batch number: 04202102001) are shown in Table 10.

TABLE 10

| Test item | Standard | | Point 0 | 3 months |
|---|---|---|---|---|
| pH value | 5.9-6.3 | | 6.0 | 6.1 |
| Osmotic molar | 270-350 | mOsmol/kg | 294 | 292 |
| concentration | | | | |
| Protein content | 45-55 | μg/ml | 51 | 51.0 |

| | | | |
|---|---|---|---|
| HPLC purity | ≥98.0% | 100.0% | 100.0% |
| In vitro biological | 50%-150% of reference | 138% | 124% |
| activity | | | |

The results for the statistical data of the accelerated stability test of JL14001 finished product at 25° C. (batch number: 04202102002) are shown in Table 11.

TABLE 11

| Test item | Standard | | Point 0 | 3 months |
|---|---|---|---|---|
| pH value | 5.9-6.3 | | 6.0 | 6.1 |
| Osmotic molar | 270-350 | mOsmol/kg | 294 | 292 |
| concentration | | | | |
| Protein content | 45-55 | μg/ml | 51 | 49.0 |

| | | | |
|---|---|---|---|
| HPLC purity | ≥98.0% | 100.0% | 100.0% |
| In vitro biological | 50%-150% of reference | 138% | 121% |
| activity | | | |

The results for the statistical data of the accelerated stability test of JL14001 finished product at 25° C. (batch number: 04202102003) are shown in Table 12.

TABLE 12

| Test item | Standard | | Point 0 | 3 months |
|---|---|---|---|---|
| pH value | 5.9-6.3 | | 6.0 | 6.2 |
| Osmotic molar | 270-350 | mOsmol/kg | 294 | 291 |
| concentration | | | | |
| Protein content | 45-55 | μg/ml | 51 | 49.0 |

| | | | |
|---|---|---|---|
| HPLC purity | ≥98.0% | 100.0% | 100.0% |
| In vitro biological | 50%-150% of reference | 138% | 124% |
| activity | | | |

The results for the statistical data of the accelerated stability test of JL14001 finished product at 25° C. (batch number: 04202102004) are shown in Table 13.

TABLE 13

| Test item | Standard | | Point 0 | 3 months |
|---|---|---|---|---|
| pH value | 5.9-6.3 | | 6.0 | 6.2 |
| Osmotic molar | 270-350 | mOsmol/kg | 294 | 291 |
| concentration | | | | |
| Protein content | 45-55 | μg/ml | 51 | 51.0 |

| | | | |
|---|---|---|---|
| HPLC purity | ≥98.0% | 100.0% | 100.0% |
| In vitro biological | 50%-150% of reference | 138% | 125% |
| activity | | | |

### 6.3 Stability Data of Other Formulas

The following formulas were treated at 50° C. for 8 days, and then subjected to SEC-HPLC and biological activity analysis. The detection results are shown in Table 14.

TABLE 14
Composition and stability data of other formulas

| | | Phosphate | Citrate | Acetate | | | | | Changes in |
|---|---|---|---|---|---|---|---|---|---|
| | Protein | buffer | buffer | buffer | | | Tween | SEC- | biological |
| Formula | concentration | system | system | system | | NaCl | 80 | HPLC | activity |
| No. | (μg/mL) | (mM) | (mM) | (mM) | pH | (mM) | (%) | (%) | (%) |
| 1 | 200 | 25 | 0 | 10 | 6 | 100 | 0.001 | 94.52 | 34.29 |
| 2 | 200 | 25 | 25 | 0 | 7 | 100 | 0.001 | 78.85 | 33.64 |
| 3 | 200 | 0 | 25 | 10 | 6 | 150 | 0.001 | 93.98 | 33.64 |
| 4 | 200 | 25 | 0 | 10 | 7 | 100 | 0.01 | 66.81 | 18.72 |
| 5 | 200 | 25 | 25 | 0 | 7 | 150 | 0.001 | 70.89 | 26.95 |
| 6 | 200 | 25 | 25 | 10 | 6 | 150 | 0.01 | 92.70 | 42.47 |
| 7 | 200 | 0 | 25 | 10 | 7 | 100 | 0.01 | 58.91 | 14.33 |
| 8 | 200 | 0 | 0 | 10 | 7 | 150 | 0.001 | 47.42 | 2.83 |
| 9 | 200 | 0 | 0 | 0 | 7 | 150 | 0.01 | 45.07 | 1.96 |
| 10 | 200 | 25 | 0 | 0 | 6 | 150 | 0.01 | 96.39 | 39.88 |
| 11 | 200 | 0 | 25 | 0 | 6 | 100 | 0.01 | 94.23 | 34.67 |
| 12 | 200 | 0 | 0 | 0 | 6 | 100 | 0.001 | 57.08 | 6.37 |
| 13 | 200 | 12.5 | 12.5 | 5 | 6.5 | 125 | 0.0055 | 86.20 | 27.94 |
| 14 | 200 | 15 | 0 | 0 | 5.5 | 125 | 0.005% | 96.72 | 38.50 |
| 15 | 200 | 25 | 0 | 0 | 5.5 | 125 | 0.005% | 94.78 | 38.90 |
| 16 | 200 | 15 | 0 | 0 | 6.5 | 125 | 0.005% | 93.29 | 44.48 |
| 17 | 200 | 25 | 0 | 0 | 6.5 | 125 | 0.005% | 92.82 | 45.59 |
| 18 | 200 | 15 | 0 | 0 | 6 | 125 | 0.005% | 96.14 | 42.57 |
| 19 | 200 | 25 | 0 | 0 | 6 | 125 | 0.005% | 95.52 | 42.42 |
| 20 | 200 | 20 | 0 | 0 | 5.5 | 125 | 0.005% | 95.75 | 40.18 |
| 21 | 200 | 20 | 0 | 0 | 6.5 | 125 | 0.005% | 93.79 | 44.28 |
| 22 | 200 | 20 | 0 | 0 | 6 | 125 | 0.005% | 95.90 | 42.84 |
| 23 | 25 | 15 | 0 | 0 | 6.1 | 140 | 0.005% | 98.99 | N/A |
| 24 | 50 | 15 | 0 | 0 | 6.1 | 140 | 0.005% | 97.85 | N/A |
| 25 | 100 | 15 | 0 | 0 | 6.1 | 140 | 0.005% | 97.74 | N/A |
| 26 | 200 | 15 | 0 | 0 | 6.1 | 140 | 0.005% | 97.26 | N/A |
| 27 | 300 | 15 | 0 | 0 | 6.1 | 140 | 0.005% | 96.08 | N/A |
| 28 | 500 | 15 | 0 | 0 | 6.1 | 140 | 0.005% | 95.69 | N/A |

All documents mentioned in the present application are incorporated by reference into the present application to the same extent as if each individual document is individually incorporated by reference. In addition, it should be understood that after reading the above teachings of the present application, those skilled in the art may make various changes or modifications to the present application, and these equivalent forms also fall within the scope defined by the claims attached to the present application.

What is claimed is:

1. A formulation comprising erythropoietin, a buffer ingredient, a surfactant and an osmotic pressure regulator, wherein the erythropoietin comprises the amino acid sequences set forth in SEQ ID NO. 1.

2. (canceled)

3. (canceled)

4. The formulation according to claim 1, wherein the pH of the formulation is about 5.5 to about 7.0.

5. (canceled)

6. The formulation according to claim 1, wherein the surfactant comprises a nonionic surfactant.

7. The formulation according to claim 1, wherein the surfactant comprises polysorbate.

8. The formulation according to claim 1, wherein the surfactant comprises polysorbate-80.

9. The formulation according to claim 1, wherein the osmotic pressure regulator comprises sodium chloride and/or mannitol.

10. The formulation according to claim 1, comprising a solvent, and the solvent comprises purified water and/or water for injection.

11. (canceled)

12. The formulation according to claim 1, wherein the content of the erythropoietin is about 25 μg/mL to about 500 μg/mL.

13. The formulation according to claim 1, wherein the content of the erythropoietin is about 50 μg/mL to about 500 μg/mL.

14. (canceled)

15. The formulation according to claim 1, wherein the buffer ingredient comprises a phosphate buffer solution, and the content of the phosphate buffer solution is about 0.001 mM to about 30 mM.

16. The formulation according to claim 1, wherein the buffer ingredient comprises a citrate buffer solution, and the content of the citrate buffer solution is about 0.001 mM to about 25 mM.

17. The formulation according to claim 1, wherein the buffer ingredient comprises an acetate buffer solution, and the content of the acetate buffer solution is about 0.001 mM to about 10 mM.

18. (canceled)

19. The formulation according to claim 1, wherein the osmotic pressure regulator comprises sodium chloride, and the content of the sodium chloride is about 100 mM to about 150 mM.

20. The formulation according to claim 1, wherein the osmotic pressure regulator comprises sodium chloride, and the content of the sodium chloride is about 5 mg/mL to about 10 mg/mL.

21. (canceled)

22. The formulation according to claim 10, comprising: 0.4 mM to 2.7 mM monobasic sodium phosphate monohydrate, 0.7 mM to 7 mM dibasic sodium phosphate, 0.01 mg/mL to 0.1 mg/mL polysorbate-80, 100 mM to 150 mM sodium chloride and 1 mL water for injection or purified water.

23. The formulation according to claim 10, comprising: 50.0 μg/mL erythropoietin, 15 mM phosphate buffer, 140 mM sodium chloride, 0.005% polysorbate-80 and water for injection or purified water.

24. The formulation according to claim 1, wherein the formulation is formulated as an injection.

25. The formulation according to claim 1, wherein the storage temperature of the formulation is about 2 to about 8° C.

26. A method for preparing the formulation according to claim 1, comprising the following steps:
1. mixing the buffer ingredient, the osmotic pressure regulator and the surfactant to obtain a mixed solution; and 2) mixing the erythropoietin with the mixed solution.

27. (canceled)

28. Use of the formulation according to claim 1 in the preparation of drugs for preventing and/or treating erythropoietin-related diseases.

29. (canceled)

30. (canceled)

31. (canceled)


SEQUENCE LISTING (1 sequence)

SEQ ID NO: 1 - protein, 165 aa, synthetic construct
  REGION 1..165: note = EPO
APPRLICDSRVLERYLLEAKEAENITTGCNETCSLNENITVPDTKVNFYAWKRMEVGQQAVEVWQGLALLSEAVLRGQALLVNSSQVNETLQLHVDKAVSGLRSLTTLLRALGAQKEAISPPDAASAAPLRTITADTFRKLFRVYSNFLRGKLKLYTGEACRTGD